(19) United States
(12) Patent Application Publication
(10) Pub. No.: US 2025/0090752 A1
(43) Pub. Date: Mar. 20, 2025

(54) CLOSED LOOP INFUSION PUMP CONTROL

(71) Applicant: ClearPoint Neuro, Inc., Solana Beach, CA (US)

(72) Inventors: Lyubomir Zagorchev, Boston, MA (US); Nathan Williams, San Diego, CA (US)

(21) Appl. No.: 18/887,829

(22) Filed: Sep. 17, 2024

Related U.S. Application Data
(60) Provisional application No. 63/583,360, filed on Sep. 18, 2023.

(51) Int. Cl. A61M 5/172; A61M 5/142; G16H 20/17

(52) U.S. Cl. CPC A61M 5/172; G16H 20/17; A61M 2005/14208

(57) ABSTRACT

A closed-loop infusion system includes a therapeutic delivery system configured to deliver a therapeutic to a patient and an imaging system configured to obtain an image of the target location. A computer processor is communicatively coupled to the therapeutic delivery system and the imaging system, wherein the computer processor is configured to control delivery of the therapeutic to the patient based on data from the imaging system.

### CROSS-REFERENCE TO RELATED APPLICATION

The present application claims priority to U.S. Provisional Application No. 63/583,360, filed on Sep. 18, 2023, and entitled “CLOSED LOOP INFUSION PUMP CONTROL,” the entirety of which is incorporated by reference herein.

### BACKGROUND

Fluid infusion systems are configured to inject a fluid, such as therapeutic fluid, into a patient. Such systems are sometimes manually controlled by a user based upon the user's review or interaction with anatomical images obtained by an imaging modality. This can result in an inexact or ineffective dose of the therapeutic to the patient. In view of this, there is a need for systems and methods for controlling the infusion of fluids to a patient based on imaging data.

### SUMMARY

Disclosed are systems and methods for controlling or managing an injection of fluid into the brain such as using imaging data. A fluid injection device or delivery device, such as a catheter, is coupled to a patient's brain such that the fluid injection device forms a fluid pathway (such as via transcranial pathway) to at least one target location within the brain. The fluid injection device is connected to a fluid transfer device, such a syringe, containing a therapeutic fluid. The syringe is further connected to a fluid pump that is configured to cause fluid to be forcibly injected into the brain via the syringe and the catheter.

The disclosed systems and methods enable the pump to be controlled manually or automatically based on imaging data. Such a configuration simplifies the workflow relative to existing systems and provides more consistent clinical results. This can result in a reduction in dosing time and can increase coverage amount, and further decrease a volume of therapeutic needed to properly treat the patient.

The details of one or more variations of the subject matter described herein are set forth in the accompanying drawings and the description below. Other features and advantages of the subject matter described herein will be apparent from the description and drawings, and from the claims.

### BRIEF DESCRIPTION OF THE DRAWINGS

FIG. 1 shows a schematic illustration of a conventional catheter and pump system.

FIGS. 2 and 3 show a catheter and pump system coupled to a diagnostic hardware and software system.

FIGS. 4 and 5 show example MRI image with brain structures outlined and therapeutic visualized.

FIG. 6 depicts a block diagram illustrating an example of a computing system consistent with implementations of the current subject matter.

### DETAILED DESCRIPTION

Before the present subject matter is further described, it is to be understood that this subject matter described herein is not limited to particular embodiments described, as such may of course vary. It is also to be understood that the terminology used herein is for the purpose of describing particular embodiments only, and is not intended to be limiting. Unless defined otherwise, all technical terms used herein have the same meaning as commonly understood by one skilled in the art to which this subject matter belongs.

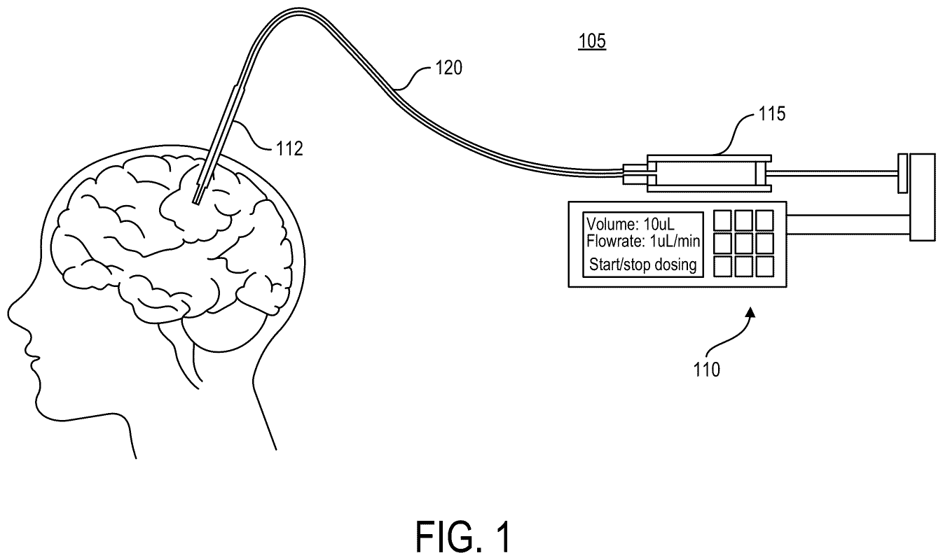

FIG. 1 is a schematic representation of a conventional catheter and pump system 105 wherein a pump assembly 110 is fluidly coupled to a patient's brain via a transcranial pathway defined by a cannula 112. The brain is shown as an example anatomical target location although any anatomical target location is within the scope of this disclosure. Thus, this disclosure is not limited to the brain being the target location for infusion. The pump assembly 110 includes a manually controlled syringe 115 that contains a fluid therapeutic. A fluid delivery device such as, for example, a catheter 120 comprising an elongated body, such as a tube with an internal lumen, provides a fluid pathway between the syringe 115 and the brain such that a user can manually control the syringe 115 to cause the syringe to expel the fluid therapeutic into the brain via the catheter 120. The pump assembly 110 can vary in configuration and can include various types of pumps. The syringe can also vary. The syringe a syringe can formed of a barrel in which a plunger assembly is slidably positioned. The barrel can have a distal coupler that can be removably attached to the catheter 120 such that the catheter 120 couples the syringe to the patient for delivering therapeutic from the syringe to the patient. The barrel defines an internal volume that can contain an initial amount of therapeutic or other substance.

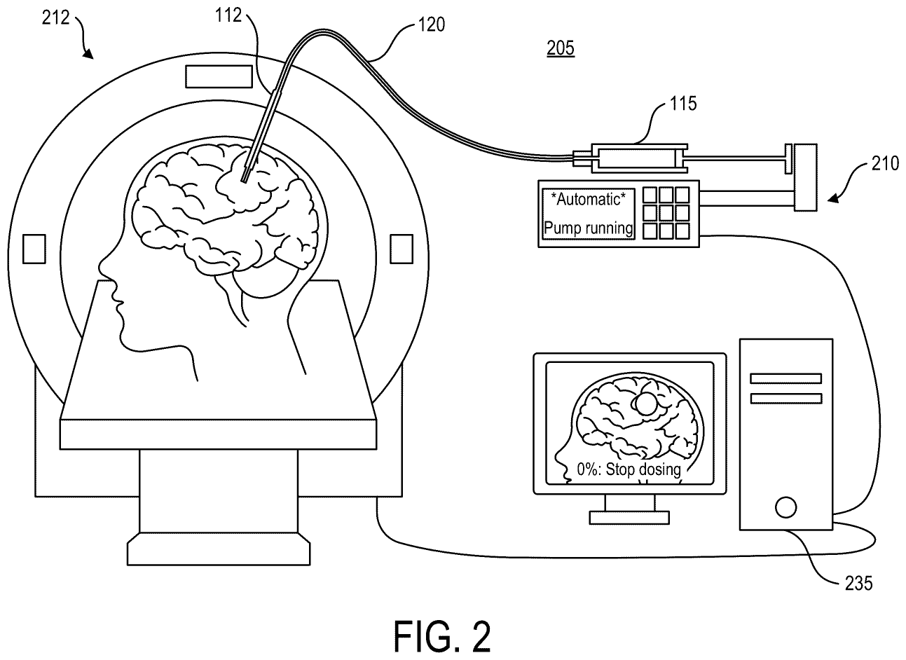

In the illustrated embodiment, the pump assembly 110 includes a screen for display any of a variety of information as well as one or more actuators, such as buttons, configured to permit the user to input predetermined dosing parameters (i.e. volume and flow rate) or manually start/stop the flow of therapeutic. The screen is configured to present a user interface for interaction by the user. In an implementation, the user interface may be disabled when the pump is being automatically controlled via the closed loop system described below with reference to FIG. 2. Alternatively, the user interface may still display information or may still allow for user interaction (e.g. start/stop, adjust flowrate) during such automatic control. The pump assembly 110 may include or be coupled to a motor for driving the pump assembly 110.

FIG. 2 schematically shows a similar catheter and pump system 205 having a therapeutic delivery system that includes pump assembly 210 and that is communicatively coupled to a diagnostic hardware and software system that includes or is coupled to an imaging system or modality (such as, for example, a magnetic resonance imaging (MRI) system 212). It should be appreciated that other imaging modality systems (such as computerized tomography (CT), Ultrasound, or live xRay/Fluoro) are within the scope of this disclosure. Moreover, the pump assembly 210 can include any type of mechanical or electromechanical mechanism for causing fluid to be administered into a body, such as any moving arm/piston mechanism, peristaltic pump or any other device or mechanism that controls the movement of fluid.

With reference still to FIG. 2, the system 205 further includes a control computer 235 that is configured to control the pump assembly 210 at least partially based on data from the diagnostic hardware and software system. The control computer 235 includes software that enables a closed loop control configuration for controlling the pump assembly 210. The diagnostic hardware and software system is configured to, in combination with the MRI system 212, image the anatomical structure (such as the brain) and monitor, as well as control, the injection of therapeutic into the brain via the pump assembly 210 at least partially based upon feedback and image data exchanged between the pump assembly and the diagnostic hardware and software system. The therapeutic of the syringe 115 may or may not include a contrast agent (such as Gadolinium in a non-limiting example) for enhanced detectability.

The closed loop control configuration may operate in real time. Alternatively, the closed loop control configuration may be delayed based wherein changes to control of the pump assembly may be delayed for a period of time after relevant data is received. The delay may be based on hardware and software limitations or may be intentionally introduced for safety or other reasons. Moreover, the system may incorporate safety limits and parameters. Such limits and parameters may be entered manually by a user or may be automatically derived from drug libraries, volume calculations or other parameters. Safety limits based on the ability of the software to detect leaked infusate, catheter backflow, and other limits may be specified by the user before infusions. For example, the system is configured to inhibit, change, stop, increase or otherwise adjust fluid flow to the patient based on whether safety limits thresholds are reached.

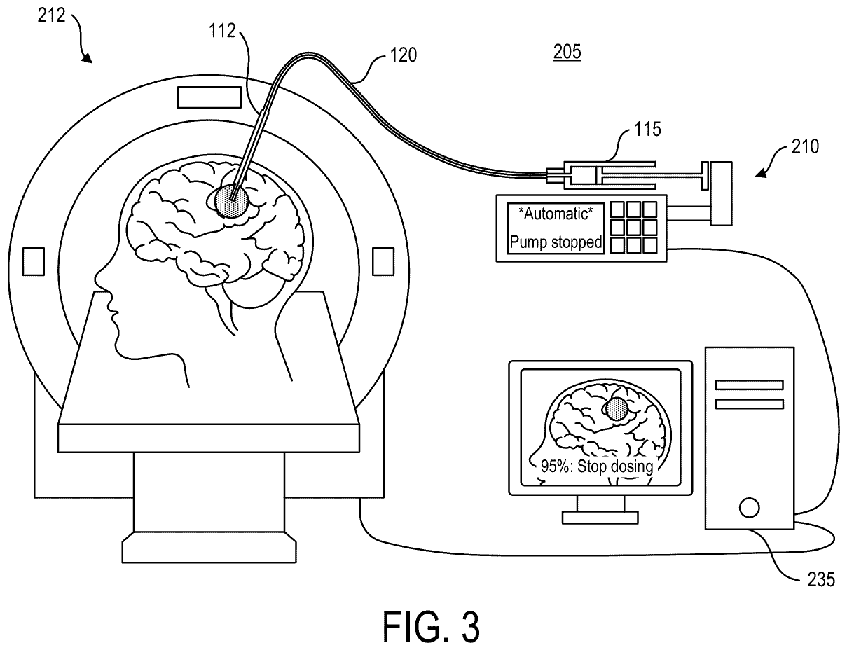

In FIG. 2, the syringe 115 of the catheter and pump system 205 is in a first or filled state wherein the syringe 115 is primed for delivery of a dose of the therapeutic to the brain. FIG. 3 shows the catheter and pump system 205 in a second state after dosing of the therapeutic via the pump assembly 210 wherein the dosing has been started, partially completed, or entirely completed. That is, at least a portion of the therapeutic has been expelled from the syringe 115 to the patient. The pump may be connected to the control computer in any of a variety if manners such as via USB, optical, wireless or any other data interface.

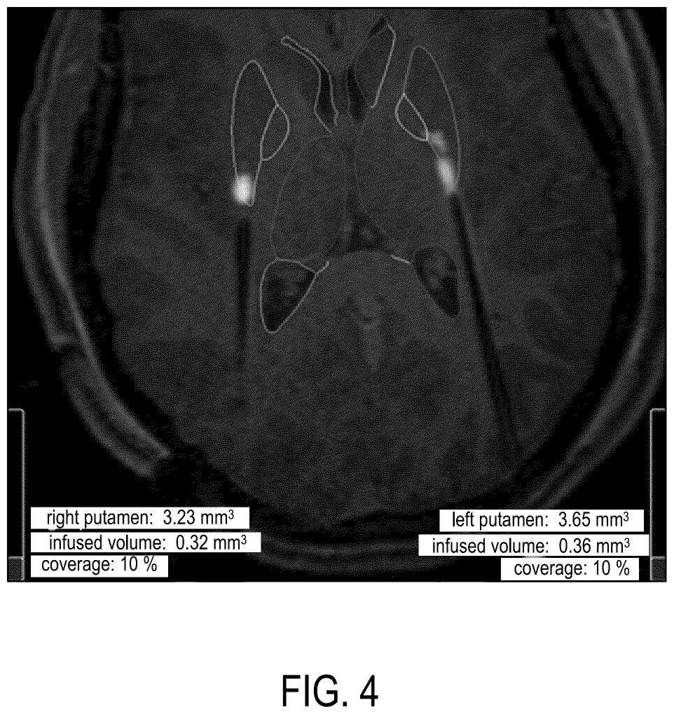

The computer 235 includes software configured to cause the pump assembly 210 or any other aspect of the system to manually or automatically identify an anatomical volume of interest in the brain based upon the image data. For example, a specific area of the brain may be identified for monitoring infusion of fluid thereto. The software is further configured to generate an image and create boundary lines on the image around at least one anatomical region or structure of interest and present an image of such boundary lines such as on a display or on a hard copy of the image. The boundary lines may be automatically generated by the software or a user can manually provide data related to the boundary lines such as by drawing boundary lines on an image. The software may also manually or automatically detect the location, amount, flow rate or other feature of therapeutic (such as a fluid therapeutic) in the brain. FIG. 4 illustrates an example MRI image with brain structures outlined and therapeutic visualized.

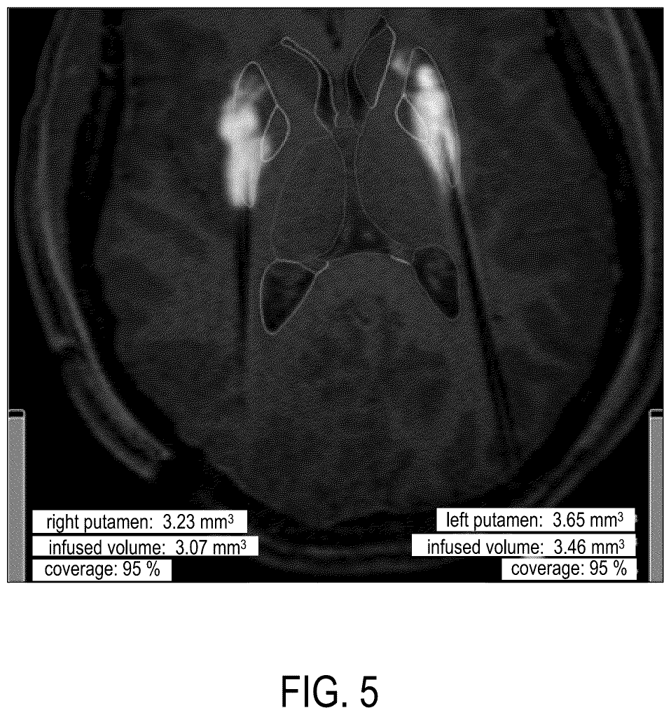

The software is further configured to cause the system, such as via an instruction via software to the computer, to calculate infused volume of therapeutic and proportion of therapeutic coverage. This information can be used to control the pump assembly 210, such as to manually (via a user command) or automatically turn the pump on, turn the pump off, or adjust the flowrate of fluid infused by the pump. FIG. 5 illustrates an MRI image with brain structure outlined and a high degree of therapeutic coverage within one or more regions of interest. The software is further configured to manually or automatically determine or calculate the coverage of therapeutic within a predetermined anatomic region that is required before the pump turns off. Such a calculation may be based on an analysis of the image data associated with the region of interest, The software may calculate the volume of therapeutic infusate outside of a targeted region to either turn off the pump or advance the catheter 120 (such as the catheter tip) to a desired location in the brain.

The software is further configured to identify other aspects of the anatomy, such as for example porosity, hydraulic conductivity, and other physical characteristics of the anatomy (e.g., in or around the region of interest) and the therapeutic. Patient-specific physical characteristics of targeted anatomy may be computed from multi-modal imaging (such as DWI, fMRI, PET) registered and fused in the space of the structural data. This information can be used to identify and modify the flow rate to provide optimized coverage, infused volume, dosing time, etc. The software is configured to cause the pump to optimize a flow rate based on imaging data, infused volume, backflow, target location or an aspect of the anatomy (such as porosity).

The software is further configured to identify optimum or enhanced dosing location of the anatomy for delivery of the therapeutic. This can be determined using physics-based simulation or geometric parameters (e.g. centroid or centroids), diffusion characteristics (e.g. porosity, viscosity) and a combination of other factors. The system can then automatically cause the catheter 120 to be positioned (such as via electromechanical components) to coincide with one or more optimum infusion locations.

If multiple target locations for delivery of therapeutic are used, then they may or may not be collinear. The system may incorporate a drive mechanism that allows for automatic adjustment of the position of the catheter in the anatomy. The dosing may be performed only at discrete points or dosing may continue concurrent to catheter movement.

The catheter 120 may vary in structure. For example, the catheter may include a single lumen opening or multiple lumen openings. The catheter may include a single lumen or multiple lumens. If the catheter includes multiple lumens then they may all be controlled together or may include individual controls per lumen.

### Example Computing System

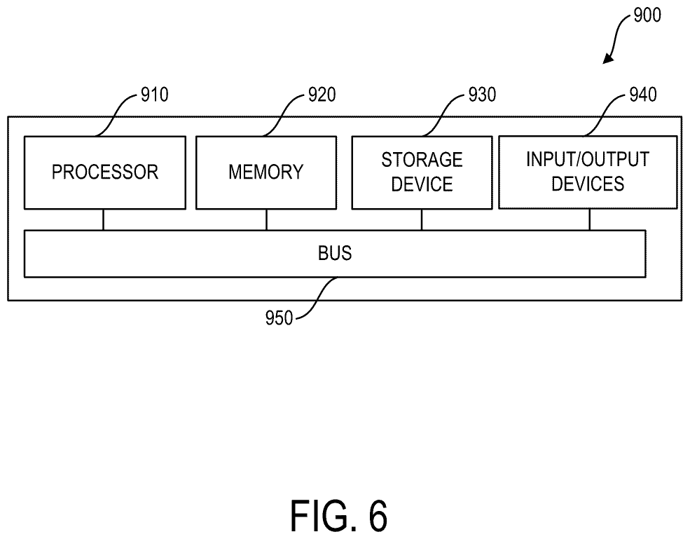

FIG. 6 depicts a block diagram illustrating an example of a computing system 900 consistent with implementations of the current subject matter. The computing system 900 may implement processes and methods described herein. The system can include or be coupled to an imaging system, an interactive user interface, and/or an inputs program to receive and manipulate physical, biometric, biomechanical, material and mechanical information and data. The system 900 can be mechanically and/or communicatively coupled to or include an imaging device such as a CT or MR device or any other imaging system. The system 900 may mechanically and/or communicatively coupled to or include a syringe system as described herein.

As shown in FIG. 6, the computing system 900 can include a processor 910, a memory 920, a storage device 930, and input/output device 940. The processor 910, the memory 920, the storage device 930, and the input/output device 940 can be interconnected via a system bus 950. The processor 910 is capable of processing instructions for execution within the computing system 900. Such executed instructions can implement one or more components of, for example, VESA, 3D-ID AI, and/or MP Tool. In some implementations of the current subject matter, the processor 910 can be a single-threaded processor. Alternately, the processor 910 can be a multi-threaded processor. The processor 910 is capable of processing instructions stored in the memory 920 and/or on the storage device 930 to display graphical information for a user interface provided via the input/output device 940.

The memory 920 is a computer readable medium such as volatile or non-volatile that stores information within the computing system 900. The memory 920 can store data structures representing configuration object databases, for example. The storage device 930 is capable of providing persistent storage for the computing system 900. The storage device 930 can be a floppy disk device, a digital cloud, a hard disk device, an optical disk device, or a tape device, or other suitable persistent storage means. The input/output device 940 provides input/output operations for the computing system 900. In some implementations of the current subject matter, the input/output device 940 includes a keyboard and/or pointing device. In various implementations, the input/output device 940 includes a display unit for displaying graphical user interfaces.

According to some implementations of the current subject matter, the input/output device 940 can provide input/output operations for a network device. For example, the input/output device 940 can include Ethernet ports or other networking ports to communicate with one or more wired and/or wireless networks, Bluetooth or digital cloud system (e.g., a local area network (LAN), a wide area network (WAN), the Internet).

In some implementations of the current subject matter, the computing system 900 can be used to execute various interactive computer software applications that can be used for organization, analysis and/or storage of data in various (e.g., tabular) format (e.g., Microsoft Excel®, and/or any other type of software). Alternatively, the computing system 900 can be used to execute any type of software application. These applications can be used to perform various functionalities, e.g., planning functionalities (e.g., generating, managing, editing of spreadsheet documents, word processing documents, and/or any other objects, etc.), computing functionalities, communications functionalities, etc. The applications can include various add-in functionalities, plug ins, or can be standalone computing products and/or functionalities. Upon activation within the applications, the functionalities can be used to generate the user interface provided via the input/output device 940. The user interface can be generated and presented to a user by the computing system 900 (e.g., on a computer screen monitor, etc.). The user interface can be integrated with other devices or virtual ecosystems.

One or more aspects or features of the subject matter described herein can be realized in digital electronic circuitry, integrated circuitry, specially designed ASICs, field programmable gate arrays (FPGAs) computer hardware, firmware, software, and/or combinations thereof. These various aspects or features can include implementation in one or more computer programs that are executable and/or interpretable on a programmable system including at least one programmable processor, which can be special or general purpose, coupled to receive data and instructions from, and to transmit data and instructions to, a storage system, at least one input device, and at least one output device. The programmable system or computing system may include clients and servers. A client and server are generally remote from each other and typically interact through a communication network. The relationship of client and server arises by virtue of computer programs running on the respective computers and having a client-server relationship to each other.

These computer programs, which can also be referred to as programs, software, software applications, applications, components, or code, include machine instructions for a programmable processor, and can be implemented in a high-level procedural and/or object-oriented programming language, and/or in assembly/machine language. As used herein, the term “machine-readable medium” refers to any computer program product, apparatus and/or device, such as for example magnetic discs, optical disks, memory, and Programmable Logic Devices (PLDs), used to provide machine instructions and/or data to a programmable processor, including a machine-readable medium that receives machine instructions as a machine-readable signal. The term “machine-readable signal” refers to any signal used to provide machine instructions and/or data to a programmable processor. The machine-readable medium can store such machine instructions non-transitorily, such as for example as would a non-transient solid-state memory or a magnetic hard drive or any equivalent storage medium. The machine-readable medium can alternatively or additionally store such machine instructions in a transient manner, such as for example, as would a processor cache or other random-access memory associated with one or more physical processor cores.

To provide for interaction with a user, one or more aspects or features of the subject matter described herein can be implemented on a computer having a display device, such as for example a cathode ray tube (CRT) or a liquid crystal display (LCD) or a light emitting diode (LED) monitor for displaying information to the user and a keyboard and a pointing device, such as for example a mouse or a trackball, by which the user may provide input to the computer. Other kinds of devices can be used to provide for interaction with a user as well. For example, feedback provided to the user can be any form of sensory feedback, such as for example visual feedback, auditory feedback, or tactile feedback; and input from the user may be received in any form, including acoustic, speech, or tactile input. Other possible input or output devices include touch screens or other touch-sensitive devices such as single or multi-point resistive or capacitive track pads, joy sticks, voice recognition hardware and software, optical scanners, optical pointers, digital image capture devices and associated interpretation software, image scanners including computer topography and magnetic resonance (MR) imaging systems and the like.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” Use of the term “based on,” above and in the claims is intended to mean, “based at least in part on,” such that an unrecited feature or element is also permissible.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

1. A closed-loop infusion system, comprising:
a therapeutic delivery system configured to deliver a therapeutic to a patient, the therapeutic delivery system including a pump assembly and a fluid delivery device, wherein the pump assembly is configured to infuse the therapeutic to a target location of a patient via the delivery catheter; and
an imaging system configured to obtain an image of the target location;
a computer processor communicatively coupled to the therapeutic delivery system and the imaging system, wherein the computer processor is configured to control delivery of the therapeutic to the patient based on feedback data from the imaging system.

2. The system of claim 1, wherein the imaging system comprises at least one of an MRI system, a CT system, an ultrasound system, and an x-ray system.

3. The system of claim 1, wherein the pump assembly includes a syringe.

4. The system of claim 1, wherein the target location includes a brain.

5. The system of claim 1, wherein computer processor monitors an injection of therapeutic to the target location.

6. The system of claim 5, wherein computer processor visualizes a location of therapeutic in the anatomy based on the image data.

7. The system of claim 1, wherein the computer processor automatically identifies an anatomical volume of interest based on data from the imaging system.

8. The system of claim 7, wherein the computer processor forms boundary lines that identify the volume of interest on an image of the target region.

9. The system of claim 1, wherein the computer processor calculates an infused volume of therapeutic to the target location.

10. The system of claim 9, wherein the computer processor controls the pump assembly based upon the infused volume.

11. The system of claim 10, wherein the computer processor automatically turns the pump assembly on or off.

12. The system of claim 10, wherein the computer processor automatically adjusts a flow rate of the therapeutic to the target location.

13. The system of claim 1, wherein the computer processor calculates an infused volume of therapeutic outside the target location.

14. The system of claim 10, wherein the computer processor causes the pump to adjust a flow rate based on imaging data, infused volume, backflow, target location or an aspect of the anatomy.

15. The system of claim 10, wherein the computer processor causes the pump to adjust a flow rate based on infused volume, backflow, target location or an aspect of the anatomy.

16. The system of claim 10, wherein the computer processor causes the pump to adjust a flow rate based on backflow, target location or an aspect of the anatomy.

17. The system of claim 10, wherein the computer processor causes the pump to adjust a flow rate based on target location or an aspect of the anatomy.

18. The system of claim 10, wherein the computer processor causes the pump to adjust a flow rate based on an aspect of the anatomy.

19. The system of claim 1, wherein the fluid delivery device is a catheter.
